(19) United States
(12) Patent Application Publication
(10) Pub. No.: US 2015/0282923 A1
(43) Pub. Date: Oct. 8, 2015

(54) VASCULAR IMPLANT RETRIEVAL METHOD, RETRIEVAL ASSEMBLY AND TOOL FOR SAME

(71) Applicant: Cook Medical Technologies LLC, Bloomington, IN (US)

(72) Inventor: Daniel J. Sirota, Bloomington, IN (US)

(21) Appl. No.: 14/714,649

(22) Filed: May 18, 2015

Related U.S. Application Data
(62) Division of application No. 13/499,062, filed on Mar. 29, 2012.
(60) Provisional application No. 61/249,633, filed on Oct. 8, 2009.

(51) Int. Cl. A61F 2/01

(52) U.S. Cl. CPC A61F 2/01; A61F 2002/011; A61F 2002/016; A61F 2002/30024

(57) ABSTRACT

A method of retrieving a vascular implant from a patient includes winding a distal segment of a retrieval tool about a vascular implant, at least in part by rotating a proximal segment of the retrieval tool, coupling the retrieval tool with the vascular implant and removing the vascular implant and the retrieval tool from the patient. The retrieval tool may include a wire having proximal, middle and distal segments. The distal segment may include a guide segment and a coupling segment, a tip and at least two turns about a longitudinal axis defined by the middle segment. The wire may further have an increasing stiffness profile in a proximal direction from the tip. A vascular implant retrieval assembly includes a vascular implant, such as a vascular filter, a retrieval tool have a distal segment wound about the vascular implant and a sheath surrounding a middle segment of the retrieval tool.

### TECHNICAL FIELD

The present disclosure relates generally to retrieval methods and mechanisms for vascular implants, and relates more particularly to retrieving a vascular implant by winding a distal segment of a retrieval tool about a vascular implant and coupling the retrieval tool therewith.

### BACKGROUND

A variety of vascular implants are well known and widely used. Vascular filters are commonly used for filtration of blood in the inferior vena cava of human patients. Stents may be used at various locations throughout a patient's vasculature to maintain or enhance blood flow where blockage has occurred. Many vascular implants are not intended for permanent placement, and may be removed after the patient's need for them has ended or upon conclusion of the service life of the vascular implant. In the case of vascular filters patients may be equipped with a vascular filter for surgical recovery or during other relatively long periods of relative immobility associated with blood clot formation. Certain stents may become less effective over time. In either case, usual protocol is removal of the vascular implant from the patient if practicable.

Different techniques and mechanisms for the removal of vascular implants from a patient have been proposed over the years. One known strategy employs a wire loop that is fed through a catheter into the patient, then positioned around a retrieval hook of a vascular implant such as a vascular filter. The wire loop may allow the vascular filter to be held and oriented while a sheath is positioned around the vascular filter to collapse and remove the same. This technique has been shown to be effective, however, there are certain situations where the vascular filter is difficult to snare and/or orient with a wire loop device.

It has been observed that endothealization of portions of certain vascular implants may occur in vivo. In general terms, endothealization is the growth of excess vascular tissue about portions of the vascular implant contacting the associated vascular wall. As a result of endothealization, portions of a vascular implant can become lodged in or against a vascular wall. This phenomenon can make it difficult to snare a hook on a vascular implant with a conventional removal device and/or inhibit proper orientation of the vascular implant for collapsing with a sheath.

### SUMMARY OF THE DISCLOSURE

In one aspect, a method of retrieving a vascular implant from a patient includes winding a distal segment of a retrieval tool about a vascular implant, at least in part by rotating a proximal segment of the retrieval tool. The method further includes coupling the retrieval tool with the vascular implant, and removing the vascular implant and the retrieval tool from the patient.

In another aspect, a vascular implant retrieval assembly includes a vascular implant, and a retrieval tool have a distal segment wound about the vascular implant, a middle segment and a proximal segment. The retrieval assembly further includes a sheath surrounding the middle segment of the retrieval tool.

In still another aspect, a retrieval tool for vascular implants includes a wire having a proximal segment, a middle segment defining a longitudinal axis and a distal segment. The distal segment includes a guide segment and a coupling segment located between the guide segment and the middle segment. The distal segment further includes a tip and at least two turns about the longitudinal axis. The wire includes an increasing stiffness profile in a proximal direction from the tip.

### BRIEF DESCRIPTION OF THE DRAWINGS

FIG. 1 is a side diagrammatic view of a retrieval tool according to one embodiment;

FIG. 2 is a pictorial view of one stage of a vascular implant retrieval procedure, according to one embodiment;

FIG. 3 is a pictorial view of another stage of the retrieval procedure;

FIG. 4 is a pictorial view of yet another stage of the retrieval procedure;

FIG. 5 is a pictorial view of yet another stage of the retrieval procedure;

FIG. 6 is a pictorial view of yet another stage of the retrieval procedure;

FIG. 7 is a partially sectioned side diagrammatic view of a vascular implant retrieval assembly according to one embodiment;

FIG. 8 is a side diagrammatic view of a retrieval tool according to another embodiment; and

FIG. 9 is an end view of the retrieval tool of FIG. 8.

### DETAILED DESCRIPTION

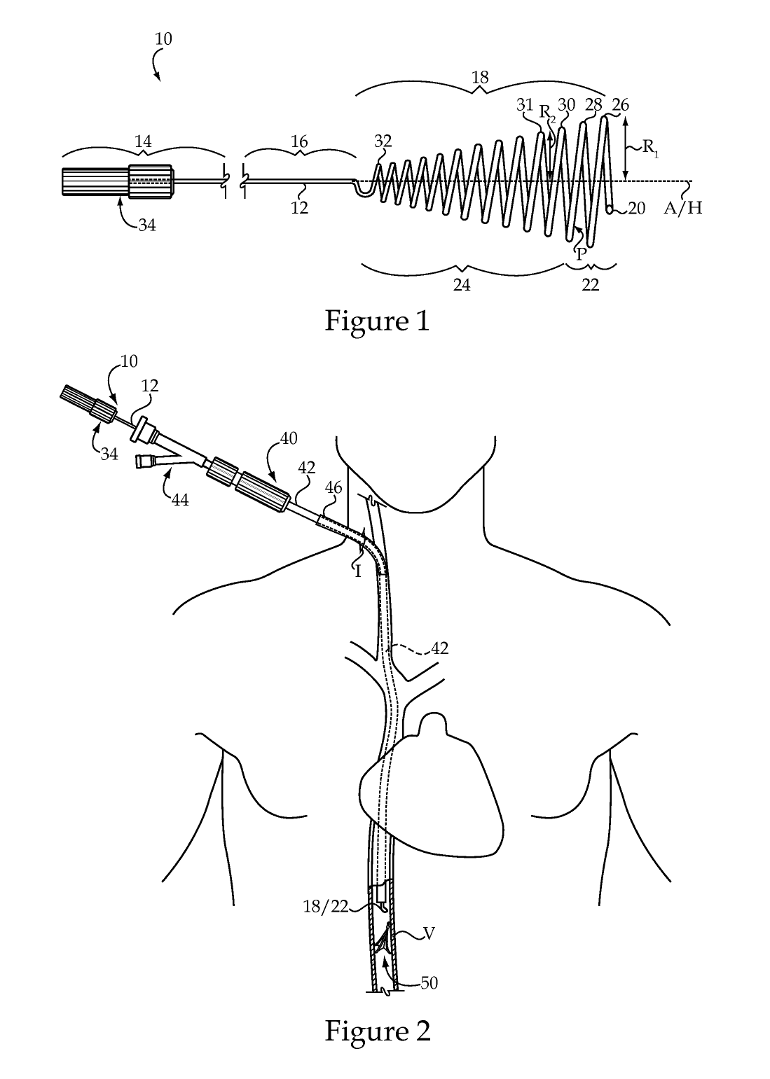

Referring to FIG. 1, there is shown a retrieval tool 10 for vascular implants according to one embodiment. Retrieval tool 10 may include a wire 12 having a proximal segment 14, a middle segment 16 and a distal segment 18. Middle segment 16 defines a longitudinal axis A. Distal segment 18 may include a guide segment 22 used in guiding retrieval tool 10 during vascular implant retrieval, as further described herein. Distal segment 18 may further include a coupling segment 24 located between guide segment 22 and middle segment 16. A handle mechanism 34 or the like may be coupled with proximal segment 14 to enable a user to manipulate proximal segment 14 of retrieval tool 10 for controlling distal segment 18 within a vascular structure of a patient.

Distal segment 18 may further include a tip 20, which is a distal end of wire 12 located opposite handle mechanism 34. Tip 20 may be configured such that abrasion or puncture risk to a vascular wall of a patient is unlikely when tool 10 is properly used. For example, tip 20 may have a hemispheric shape or another shape devoid of sharp edges and surface non-uniformities. Grinding, polishing and other manufacturing techniques to avoid sharp edges, surface non-uniformities, etc., in wire tips are known in the art. Tip 20 might also include a ball positioned thereon or a relatively short section of wire 12 folded back on itself to form a loop. As further explained herein, tip 20 and adjacent portions of guide segment 22 may also be “soft,” having a relatively low stiffness as compared with other portions of wire 12.

Distal segment 18 may further include at least two turns about longitudinal axis A. In the illustrated embodiment, distal segment 18 includes a first turn 26 and a second turn 28, and a plurality of additional turns 30, 31 and 32. Turns 26 and 28 may be part of guide segment 22, whereas turns 30, 31 and 32 may be part of coupling segment 24. Turns 26 and 28 may be understood as wire guiding turns and turns 30-32 may be understood as implant coupling turns, as further described herein. No specific number of turns is required, however in the illustrated embodiment a total of approximately twelve turns is used. In FIG. 1, reference numerals have been omitted from certain of the turns located between turn 31 and turn 32 for purposes of clarity. It may further be noted from FIG. 1 that the relative size of turns 26-32 is non-uniform. In particular, a turn radius R₁ defined by turn 26 relative to longitudinal axis A is relatively greater than a turn radius R₂ defined by turn 30 relative to longitudinal axis A. Turn radii of turns 26-32 relative to longitudinal axis A may be progressively smaller in a proximal direction as shown. The illustrated configuration represents one practical implementation strategy in which a tapered shape of distal segment 18 is used. The present disclosure is not thereby limited, however, as further discussed herein.

In one embodiment, distal segment 18 may include a spring wire having a rest shape defining a helix which includes turns 26-32. The spring wire may comprise a portion of distal segment 18 including a pseudoelastic or superelastic alloy, as further described herein. Accordingly, distal segment 18 may be deformed under a load to, or nearly to, a linear configuration, but may tend to assume its rest shape when the load is removed. The helix defined by distal segment 18 in the biased configuration may include a helix axis H collinear with longitudinal axis A. Where distal segment 18 is tapered, the helix defined by distal segment 18 may include a distally expanding conical taper. The term distally expanding conical taper should be understood to mean that a shape defined by distal segment 18 includes a cone having a diameter that is progressively larger in a direction towards tip 20. Other embodiments are contemplated where non-helical shapes, non-conical shapes, and non-tapered shapes are used.

In general, it will be desirable to allow a user of retrieval tool 10 to manipulate proximal segment 14 via handle mechanism 34, such as by rotating handle mechanism 34 or sliding handle mechanism 34 in a proximal or distal direction, and transmit said manipulation to distal segment 18. Thus, a user may rotate handle mechanism 34 and thereby induce rotation of distal segment 18. In one embodiment, rotation of handle mechanism 34 one complete turn will induce a rotation of distal segment 18 one complete turn. In other embodiments, twisting or flexing of wire 12 between handle mechanism 34 and distal segment 18 could result in one complete turn of handle mechanism 34 inducing less than one complete turn of distal segment 18. To enable the transfer of torque from handle mechanism 34 to more distally located portions of retrieval tool 10 wire 12 may be made relatively stiff for a majority of its length. To enable use of tip 20 and other portions of distal segment 18 within a vascular structure of a patient in a desired manner, however, more distal portions of wire 12 may be relatively less stiff. To this end, wire 12 may include an increasing stiffness profile in a proximal direction from tip 20. The term increasing stiffness profile is intended to mean that an average stiffness of wire 12 increases in a direction from tip 20 towards proximal segment 14. The transition from a relatively lesser stiffness to a relatively greater stiffness in a proximal direction might be either gradual or abrupt. Thus, stiffness of wire 12 might change abruptly from a relatively low stiffness distally of a given point P to a relatively higher stiffness proximally of the given point P. Alternatively, stiffness could change gradually and even linearly along a minority or a majority of a length of wire 12. In one embodiment, at least one of turns 26 and 28 of guide segment 22 may have a relatively lesser stiffness whereas at least one of turns 30-32 may have a relatively greater stiffness. Middle segment 16 and proximal segment 14 may also each have a stiffness greater than distal segment 18. In light of the foregoing description it will be understood that an increasing stiffness profile may be associated with wire 12 in various different instances.

It will further be recalled that wire 12 may include a spring wire, and retrieval tool 10 may have a rest shape corresponding approximately to the configuration illustrated in FIG. 1. The rest shape may be a shape that wire 12 tends to assume when no external load is applied, in contrast to a refracted shape assumed when wire 12 is constrained within a sheath, etc. To this end, wire 12, in particular portions of wire 12 located in distal segment 18, may be pseudoelastic or superelastic. Suitable metal alloys are commercially available under the trade name NITINOL®. Other known alloys having suitable characteristics are commercially available under the trade name ELGILOY®. Stiffness in wire 12 may be tailored by way of known manufacturing techniques, such as certain heat treatment techniques to achieve spatial variation in the stiffness of a pseudoelastic or superelastic alloy wire. It should be understood, however, that the present disclosure is not limited to the use of any particular materials or manufacturing techniques.

Turning now to FIG. 8, there is shown a retrieval tool 110 according to another embodiment. Retrieval tool 110 has certain similarities with retrieval tool 10 described above, and may be made with the use of similar materials and by way of similar manufacturing techniques, but has certain differences. Retrieval tool 110 may include a wire 112 including a distal segment 118 having a guide segment 122 whereupon a tip 120 is located, and an implant coupling segment 124 positioned proximally of guide segment 122. Wire 112 may further include a middle segment 116, and a proximal segment (not shown) configured similarly to proximal segment 14 of retrieval tool 10 and coupled with a handle mechanism (not shown). Middle segment 116 may define a longitudinal axis B, and distal segment 118 may include a plurality of turns about longitudinal axis B. In the embodiment shown, and as further depicted in FIG. 9, the one or more turns of distal segment 118 may lie within a common plane oriented normal to longitudinal axis B. Distal segment 118 may further include an implant coupling notch 125, as further described herein. Wire 112 may also include an increasing stiffness profile in a proximal direction from tip 120, similar to wire 12. It should be appreciated that retrieval tool 110 may be used in a manner analogous in many ways to retrieval tool 10. Accordingly, discussion herein of the configuration, construction and use of retrieval tool 10 should be understood to refer similarly to retrieval tool 110, except where otherwise indicated.

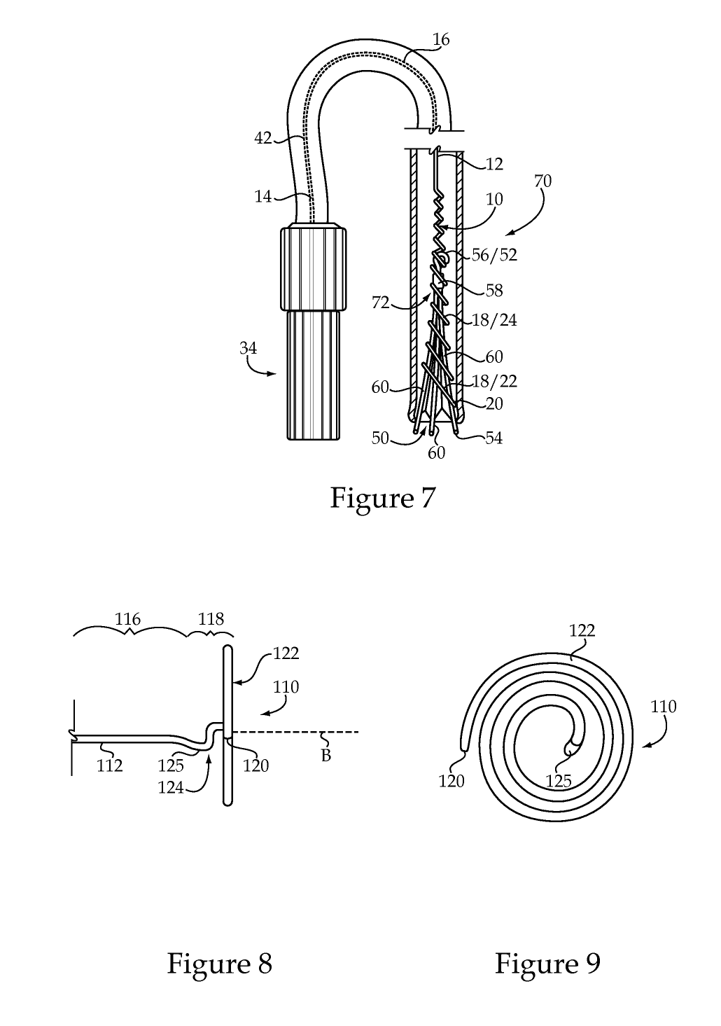

It will be recalled that retrieval tool 10 may be used in retrieving a vascular implant from a patient. Vascular implants amenable to retrieval according to the methods and apparatuses of the present disclosure may include vascular filters of the type commonly used to filter blood clots. Such vascular filters are commonly positioned in the inferior vena cava of a patient. The present disclosure is not thusly limited, however, and other vascular implants such as certain stents and the like may be retrieved in accordance with the teachings set forth therein. Turning now to FIG. 7, there is shown a vascular implant retrieval assembly 70 as it might appear just prior to or following removal from a patient. Retrieval assembly 70 may include a vascular implant 50, retrieval tool 10 and a sheath 42 surrounding portions of retrieval tool 10 and vascular implant 50 and holding vascular implant 50 in a collapsed configuration. In particular, sheath 42 may surround middle segment 16, and may also at least partially surround distal segment 18. It may be noted from FIG. 7 that distal segment 18 is wound about vascular implant 50. In one embodiment, retrieval tool 10, and in particular distal segment 18, may be wound about vascular implant 50 a plurality of times. Vascular implant 50 may include an expanded configuration, comprising a use configuration, and a collapsed configuration comprising a retrieval configuration as shown in FIG. 7 when positioned at least partially within sheath 42.

Certain features of vascular implant 50 are also evident in FIG. 7. In particular, vascular implant 50 may include a first end 52 positioned proximally in FIG. 7, and a second end 54 positioned distally in FIG. 7. A hook 56 may be located at first end 52. First end 52 may further include a hub end where a hub 58 is located. A plurality of struts 60 may extend between first end 52 and second end 54. Vascular implant 50 is similar to vascular implants of the type available from the Assignee of the present patent application, and known under the trade name GUNTHER TULIP™ for use in the prevention of recurrent pulmonary embolism via placement in the inferior vena cava. As alluded to above, however, other filters and other vascular implants are amenable to retrieval according to the teachings of the present disclosure. As will be further apparent from the following description, retrieval tool 10 may be used to couple with vascular implant 50 for collapsing via sheath 42 and eventual removal from a patient. To this end, distal segment 18 may include a coupling 72 such as a frictional or other mechanical coupling with vascular implant 50 which enables snaring or otherwise grabbing vascular implant 50 with retrieval tool 10.

### INDUSTRIAL APPLICABILITY

Referring to FIG. 2, there is shown a retrieval tool 10 in use with a sheath system 40 at one stage of a vascular implant retrieval procedure. A vascular implant 50, which may be a vascular filter as described above, is shown positioned within an inferior vena cava V of a patient. Sheath system 40 may include a sheath 42 extending from a proximal location outside of the patient's body to a distal location close to vascular implant 50. The terms distal and proximal are used in the following description in a manner consistent with that used above in connection with features of retrieval tool 10. A Y-fitting 44 may be a component of sheath system 40 to enable the introduction of contrast agents or the like, or for other known purposes. Sheath system 40 may also include a pin vise for fixing retrieval tool 10 relative to certain components of sheath system 40 when desired. An access or guide sheath 46 may be used in a conventional manner to provide access for sheath system 40, retrieval tool 10 and other mechanisms, via an incision I. Preparation for removal of a vascular implant from a patient according to the present disclosure may take place in a conventional manner, including forming incision I, passing access sheath 44 and/or a dilator or the like through incision I and introducing a wire guide into the patient. Access to the vena cava V may take place via the right jugular vein, but alternatives such as access via the left jugular vein are contemplated.

It should be appreciated that various different sheath systems and wire guides might be used, and the present disclosure is not limited to any particular strategy. In one practical implementation, sheath system 40 may include a single sheath 42 introduced into the patient by sliding over a wire guide once positioned appropriately in the vena cava V. Dual coaxial sheaths may also be used. Once one or more sheaths are positioned as desired, the wire guide may be removed, and retrieval tool 10 inserted into sheath 42. The presently described procedure will typically take place with the assistance of radiography. The mechanisms used such as sheath 42, tool 10 and the wire guide will typically be radiopaque. It may also be desirable for a contrast agent to be used to enhance the ability of a physician or technician to visually monitor progress of the procedure via an electronic display. To this end, a flush catheter or the like may be passed through sheath 42 prior to introducing retrieval tool 10, and a contrast agent injected into the vena cava V. Y-fitting 44 may also be used to introduce a contrast agent prior to or during the implant retrieval procedure.

In FIG. 2, retrieval tool 10 is shown as it might appear after being passed into the patient via sheath 42. Handle 34 is positioned at a location outside the patient where it may be manipulated by a user, and guide segment 22 protrudes slightly out of sheath 42 into the vena cava V. In one embodiment, sheath 42 is positioned as desired within the patient, and then retrieval tool 10 is fed through sheath 42. The present disclosure is not thereby limited, however, and in other instances sheath 42 and retrieval tool 10 might be passed through an outer sheath and introduced into the patient as a preassembled package. Retrieval tool 10 will typically be made available as a sterile package including handle mechanism 34 and wire 12, and could also be provided preassembled with or otherwise packaged with a suitable sheath.

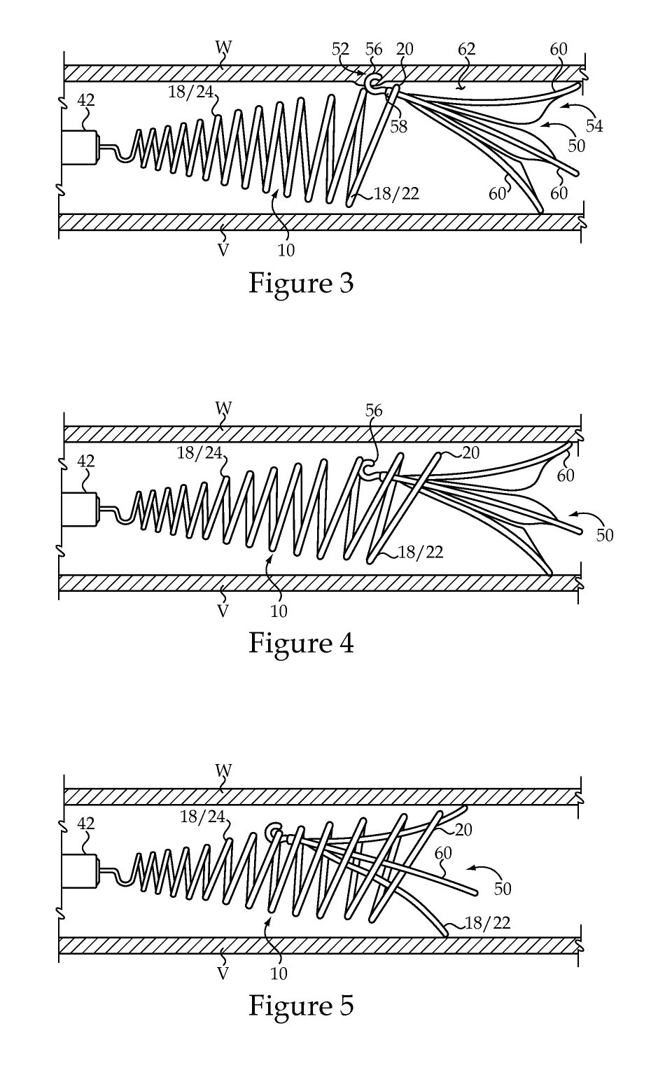

As mentioned above, in FIG. 2 guide segment 22 is protruding from sheath 42 within the vena cava V. Sheath 42 has been positioned relatively close to vascular implant 50 such that a user can manipulate handle mechanism 34 to extend retrieval tool 10 and engage vascular implant 50 for retrieval as further described herein. In one embodiment, retrieval tool 10 may be extended sufficiently from sheath 42 to assume its rest, use configuration prior to engaging with vascular implant 50. In other instances, however, retrieval tool 10 may be only partly extended from sheath 42 when engagement with vascular implant 50 commences, and further extended as the retrieval procedure proceeds. Referring also to FIG. 3, there is shown retrieval tool 10 extended from sheath 42 such that distal segment 18 has assumed the rest configuration defining a helix as described herein. From the stage depicted in FIG. 2 a user may advance retrieval tool 10 in an axial direction to extend distal segment 18 from sheath 42 while maintaining sheath 42 substantially stationary within the vena cava V. Alternatively, sheath 42 may also be advanced in an axial direction while axially advancing retrieval tool 10. In either event, once tip 20 has been positioned close to vascular implant 50, the process of engaging with and capturing vascular implant 50 with retrieval tool 10 may begin.

In FIG. 3, tip 20 of retrieval tool 20 is shown just beginning to pass into a gap 62 defined by vascular implant 50 and a vascular wall W of the patient. It may be noted that hook 56 of vascular implant 50 is positioned against, and may be lodged in, vascular wall W. It has previously been recognized that certain parts of vascular implants may become endothealized over time, resulting in lodging against or within vascular tissue of a patient. This phenomenon may create difficulty in removing the vascular implant by way of traditional techniques. In FIG. 3, endothealization of hook 56 and adjacent portions of vascular implant 50 can create an impediment to removing vascular implant 50 with certain conventional techniques, as purchase on vascular implant 50 and proper orienting of vascular implant 50 for removal may be difficult to attain. Previously, it was not uncommon for endothealized vascular implants to be left in a patient.

Distal segment 18 may be used to dislodge the endothealized portion of vascular implant 50 from vascular wall W. As mentioned above, in FIG. 3 tip 20 is shown as it might appear just beginning to enter gap 62. It will be recalled that guide segment 22 may be relatively flexible. Guide segment 22 may also have an outer diameter, the diameter of the helix defined thereby, that is equal or nearly equal to an inside diameter of vena cava V. By manipulating proximal segment 14 with handle mechanism 34, a user can rotate guide segment 22 and/or adjust guide segment 22 in proximal or distal directions to seek out gap 62 with tip 20. With the assistance of radiography, relatively minute adjustments with handle mechanism 34 may be used to work guide segment 22 into an orientation and location where tip 20 can begin to pass into gap 62. The relative flexibility of guide segment 22 and configuration of tip 20 allows a user to slide tip 20 along an inside diameter of the vena cava V to search for gap 62 without undue risk of abrading or puncturing wall W.

Referring also to FIG. 4, there is shown retrieval tool 10 having been wound about vascular implant 50 a plurality of times, and with vascular implant 50 dislodged from vascular wall W. Distal segment 18 has been advanced in a distal direction as compared with FIG. 3. Advancing distal segment 18 in a distal direction may take place during winding guide segment 22 about vascular implant 50, but could occur as a discrete step. In particular, guide segment 22 has been wound about hub end 52, and then wound about struts 60. With retrieval tool 10 wound about vascular implant 50, a user can manipulate retrieval tool 10 to pull the endothealized portion of vascular implant 50 away from wall W. It is contemplated that rotation of retrieval tool 10 and/or motion of retrieval tool in a distal, proximal or back and forth distal-proximal direction may be used to urge the endothealized portion of vascular implant away from vascular wall W. As winding of distal segment 18 about vascular implant 50 progresses, the conical taper of distal segment 18 can enable contact between distal segment 18 and vascular implant 50 to exert an increasing force pulling vascular implant 50 away from wall W. This is so because progressively smaller turns of distal segment 18 may be wound into engagement with vascular implant 50 close to the endothealized portion. Similarly, progressively stiffer turns of distal segment 18 may be wound into engagement with vascular implant 50 close to the endothealized portion. Thus, subsequent to guiding tip 20 into gap 62, a user may dislodge the endothealized portion of vascular implant 50 with retrieval tool 10. The number of times it is necessary to wind distal segment 18 about vascular implant 50 to enable dislodging vascular implant 50 may vary. For a more extensive endothealization, it may be necessary to wind retrieval tool 10 about vascular implant 50 several times. For less extensive endothealization, dislodging of vascular implant 50 may be possible after only one or two complete turns of retrieval tool about vascular implant 50. To prepare for dislodging the endothealized portion of vascular implant 50 via guiding tip 20 through gap 62 proximal segment 14 will typically be rotated at least one complete turn. Coupling of coupling segment 24 with vascular implant 50 as further described herein will typically include rotating proximal segment 14 at least one complete additional turn.

Referring also to FIG. 5, there is shown retrieval tool 10 having been wound about vascular implant 50 an additional plurality of times as compared with FIG. 4. It may also be noted from FIG. 5 that guide segment 22 has become entwined among struts 60. It will be recalled that coupling segment 24 may be relatively stiffer than guide segment 22. In FIG. 4, relatively stiffer coupling segment 24 has been positioned in contact with portions of vascular implant 50 including hook 56, hub 58 and portions of struts 60. The relatively greater stiffness and tightening of the helix of coupling segment 24 allows grabbing of vascular implant 50 to establish coupling 72 therewith. Coupling retrieval tool 10 with vascular implant 50 in the configuration shown in FIG. 5 may further be understood as establishing a purchase on vascular implant 50 with retrieval tool 10. It will be recalled that coupling segment 24 may have a relatively greater coefficient of friction than guide segment 22. The different friction coefficients may be the result of different coatings on the respective segments, such as a relatively high friction coating on coupling segment 24 and a relatively low friction coating on guide segment 22. The different friction coefficients could also be the result of different surface treatments such as polishing of guide segment 22 and roughening or no polishing of coupling segment 24.

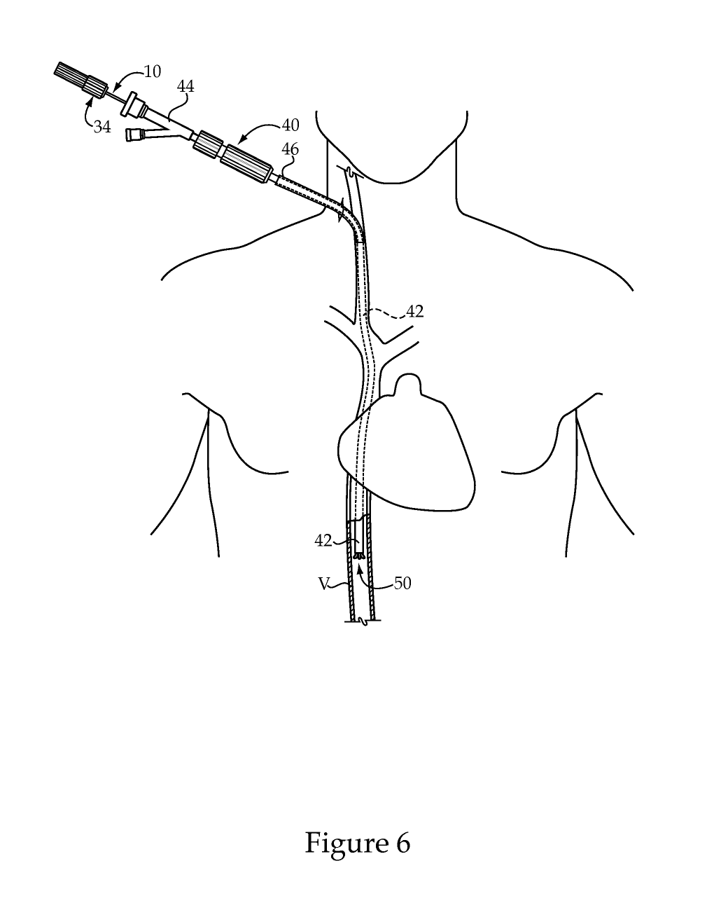

Once a purchase on vascular implant 50 with retrieval tool 10 is established, sheath 42 may be advanced in a distal direction relative to vascular implant 50 and retrieval tool 10. Coupling of vascular implant 50 with retrieval tool 10 may resist displacing vascular implant 50 during advancing sheath 42. As noted above, a pin vise or other mechanism may be used to hold retrieval tool 10 and vascular implant 50 in place during advancing sheath 42. FIG. 5 shows sheath 42 having been advanced in a distal direction toward vascular implant 50. From the configuration shown in FIG. 5, sheath 42 may be advanced further to surround and collapse vascular implant 50 within sheath 42. Referring also to FIG. 6, there is shown sheath 42 having been advanced to at least partially surround vascular implant 50 and position vascular implant 50 for removal from the patient. Once vascular implant 50 is collapsed via sheath 50, the retrieval assembly including sheath 42, vascular implant 50 and retrieval tool 10 may be removed from the patient while sheath 42 is positioned about vascular implant 50.

Returning to FIGS. 8 and 9, retrieval tool 110 may be used in a manner similar to that described herein in connection with retrieval tool 10. It will be recalled that retrieval tool 110 may include an implant coupling notch 125. Notch 125 may be used to couple with a hook such as hook 56 or another part of a vascular implant. Winding of retrieval tool 110 about vascular implant 50, or another vascular implant, may thus include winding guide segment 122 about a vascular implant one or more times, and dislodging an endothealized portion of the vascular implant. Retrieval tool 110 may then be advanced in a distal direction and further rotated until notch 125 can engage with hook 56 or another part of the subject vascular implant. Engagement with notch 125 may be used to resist displacing the vascular implant during advancing a sheath.

The present description is for illustrative purposes only, and should not be construed to narrow the breadth of the present disclosure in any way. Thus, those skilled in the art will appreciate that various modification might be made to the presently disclosed embodiments without departing from the full and fair scope and spirit of the present disclosure. Other aspects, features and advantages will be apparent upon an examination of the attached drawings and appended claims.

1. A method of retrieving a vascular implant from a patient comprising the steps of:
winding a distal segment of a retrieval tool about a vascular implant, at least in part by rotating a proximal segment of the retrieval tool;
coupling the retrieval tool with the vascular implant; and
removing the vascular implant and the retrieval tool from the patient at least in part by moving the vascular implant at least partially into a sheath with the retrieval tool.

2. The method of claim 1 further comprising a step of guiding the distal segment of the retrieval tool into a gap defined by the vascular implant and a vascular wall of the patient, at least in part by rotating the proximal segment of the retrieval tool.

3. The method of claim 2 further comprising a step of dislodging an endothealized portion of the vascular implant from the vascular wall with the retrieval tool, subsequent to the guiding step.

4. The method of claim 1 further comprising the steps of:
advancing a sheath relative to the vascular implant to position the sheath about the vascular implant; and
resisting displacing the vascular implant during advancing the sheath at least in part via the coupling step;
wherein the step of removing includes removing the vascular implant while the sheath is positioned about the vascular implant.

5. The method of claim 1 wherein the step of winding includes rotating the proximal segment of the retrieval tool at least one complete turn and the step of coupling includes rotating the proximal segment of the retrieval tool at least one complete additional turn.

6. The method of claim 5 wherein the step of coupling further includes establishing a purchase on the vascular implant with the retrieval tool.

7. The method of claim 6 wherein:
the step of winding further includes winding a guide segment of the distal segment about a hub end of the vascular implant, then winding the guide segment about a plurality of struts of the vascular implant extending between the hub end and a second end of the vascular implant; and
the step of coupling further includes establishing a purchase on the plurality of struts with a coupling segment of the distal segment which is positioned proximally of the guide segment.

8. The method of claim 5 further comprising a step of advancing the distal segment of the retrieval tool in a distal direction during the step of winding.

9. The method of claim 8 wherein the step of advancing the distal segment of the retrieval tool further includes advancing a guide segment having a relatively lesser stiffness and including at least one turn defining a relatively greater turn radius, and advancing a coupling segment having a relatively greater stiffness and including at least one additional turn defining a relatively lesser turn radius.

10. A vascular implant retrieval assembly comprising:
a vascular implant;
a retrieval tool having a distal segment wound about the vascular implant, a middle segment and a proximal segment; and
a sheath surrounding the middle segment of the retrieval tool.

11. The retrieval assembly of claim 10 wherein:
the distal segment of the retrieval tool includes a guide segment having a tip, and a coupling segment positioned proximally of the guide segment and including a coupling with the vascular implant; and
the retrieval tool defines a longitudinal axis, the guide segment including at least one turn about the longitudinal axis and the coupling segment including at least one additional turn about the longitudinal axis.

12. The retrieval assembly of claim 11 wherein the retrieval tool includes an increasing stiffness profile in a proximal direction from the tip.

13. The retrieval assembly of claim 11 wherein the distal segment includes a spring wire having a retracted shape and a rest shape defining a helix which includes the at least one turn and the at least one additional turn.

14. The retrieval assembly of claim 13 wherein the helix includes a distally expanding conical taper.

15. The retrieval assembly of claim 11 wherein:
the vascular implant includes a vascular filter having an expanded configuration and a collapsed configuration, the vascular implant further having a first end which includes a hub end, a second end, and a plurality of struts extending between the first end and the second end;
the coupling segment is wound about the plurality of struts; and
the sheath surrounds at least a portion of the vascular implant and holds the vascular implant in the collapsed configuration.

16.-20. (canceled)

21. A vascular implant retrieval assembly comprising:
a vascular implant;
a retrieval tool having a distal segment, a middle segment and a proximal segment; and
a sheath surrounding the middle segment of the retrieval tool; and
the vascular implant retrieval assembly having a pre-engagement configuration in which the vascular implant is out of contact with the retrieval tool, and a post-engagement configuration in which the distal end of the retrieval tool is wound about the vascular implant and at least a portion of the vascular implant is received into the sheath.

22. The retrieval assembly of claim 21 wherein the distal segment includes a spring wire having a retracted shape and a rest shape defining a helix which includes the at least one turn.

23. The retrieval assembly of claim 22 wherein the helix includes a distally expanding conical taper.

24. The retrieval assembly of claim 23 including a handle mechanism that is textured for hand manipulation attached to the proximal segment of the retrieval tool and being positioned outside of the sheath.

25. The retrieval assembly of claim 24 wherein the vascular implant is a vascular filter.
